(19) United States
(12) Patent Application Publication
(10) Pub. No.: US 2010/0323432 A1
(43) Pub. Date: Dec. 23, 2010

(54) SAMPLE PROCESSING DEVICE FOR MICROCHIP

(75) Inventors: Minoru Asogawa, Tokyo (JP); Hisashi Hagiwara, Kanagawa (JP); Tohru Hiramatsu, Nagano (JP)

Correspondence Address: YOUNG & THOMPSON, 209 Madison Street, Suite 500, Alexandria, VA, 22314, US

(73) Assignees: NEC CORPORATION, Tokyo (JP); AIDA ENGINEERING, LTD., Sagamihara-shi, Kanagawa (JP)

(21) Appl. No.: 12/677,419

(22) PCT Filed: Sep. 5, 2008
(86) PCT No.: PCT/JP2008/066477

§ 371 Date: Mar. 10, 2010

(30) Foreign Application Priority Data
Sep. 10, 2007 (JP) 2007-233574

(51) Int. Cl. C12M 1/34; B01L 3/00

(52) U.S. Cl. 435/287.2; 422/504

(57) ABSTRACT

A sample processing device for a microchip, including: a sample vessel for packing a sample therein; and a reaction vessel which is continuous with the sample vessel through a channel, and to which the sample is sequentially delivered to be packed and mixed therein, in which the sample is repeatedly delivered between the sample vessel and the reaction vessel through the channel so that the sample is stirred and mixed.

### TECHNICAL FIELD

This invention relates to a sample processing device for a microchip, including a plurality of reaction vessels and reagent vessels used for extraction, analysis, and the like of a micro component such as a gene, in which the reaction vessels and the reagent vessels are continuous with each other through a micro channel.

### BACKGROUND ART

In recent years, as described in Japanese Unexamined Patent Application Publication (JP-A) No. 2003-248008 A (Patent Document 1) and Japanese Unexamined Patent Application Publication (JP-A) No. 2006-55025 A (Patent Document 2), a mechanism for stirring a sample and reaction solution packed in a minute-volume vessel in extraction and analysis of a gene and a nucleic acid.

Further, a technology of reacting and analyzing an extremely minute volume of several 1 μL of sample, which is called a microchip is described in Branejerg et al., “Fast Mixing by Lamination”, Proc. IEEE Micro Electro Mech. Syst. Conf. (MEMS '96), pp. 441-446, (1996). (Non-patent Document 3), Mengeaud et al., “Mixing Steps in a Zigzag Microchannel: Finite Element Simulations and Optical Study”, Analytical Chemistry, vol. 74, no. 16, pp. 4279-4286, (2002). (Non-patent Document 4), Jia-Kun et al., “Electroosmotic flow mixing in zigzag microchannels”, Electrophoresis, vol. 28. no. 6. pp. 975-983, (2007). (Non-patent Document 5).

Specifically, Patent Document 1 described above discloses a mechanism, in which, for “stirring a reaction solution by imparting magnetic field variation from the exterior of a reaction vessel to magnetic beads contained in the reaction solution”, a plurality of electromagnets are revolved on the reaction vessel, and the electromagnets are sequentially excited so as to circulate and move the magnetic beads in the reaction vessel by a magnetic force, as a result of which the reaction solution in the reaction vessel is stirred and mixed. Further, in Patent Document 1, as an embodiment, it is described that “the reaction vessel has a size of about 20 mm×60 mm, its thickness is about 0.2 mm and volume is about 250 μL”.

Further, in Patent Document 2 described above, it is described that “micro heaters provided in the micro reaction vessel are continuously pulse-heated and the reaction solution is stirred by expansion and condensation of produced bubbles”.

### DISCLOSURE OF THE INVENTION

### Problems to be Solved by the Invention

However, in the conventional technology disclosed in Patent Document 1 described above, though the plurality of electromagnets are required to be placed in the reaction vessel, it is impossible to place them in the reaction vessel having an extremely minute volume of several μL. Further, the conventional technology disclosed in Patent Document 1 has the following problems: a complicated control mechanism for sequentially exciting the plurality of electromagnets, and hence the size thereof is large for a means for stirring the reaction vessel in the microchip, and electrical power consumption also becomes large.

Further, the conventional technology disclosed in Patent Document 2 described above, bubbles are produced in the reaction solution by the heaters provided in the reaction vessel, and the reaction solution is stirred by action of a force generated by expansion and condensation of the bubbles. However, there are following problems: the function of the sample and the reaction solution is deteriorated due to the air generated as a form of bubbles and a temperature increase due to the heaters; and a difficult control of controlling a production amount of the bubbles is required. Further, there is also a problem in that heaters to be stored in the extremely-minute-volume reaction vessel of several μL and a control mechanism for performing proper temperature control are required, and hence the device is complicated and enlarged.

Further, in the conventional technology disclosed in Non-patent Document 3, the solution is stirred by providing in a sterically-intersecting manner two channels in which two types of solutions flow, and by repeating mixing and separation of the solution. However, it is not easy to arrange the two channels sterically with high accuracy. Further, in order to sufficiently stirring the solution, it is required to sterically provide a large number of intersection-arrangement portions, and hence the size becomes spatially large. In addition, a stirred object is produced after flowing through the intersectionally-arranged channels, and hence samples to be flowed are required more than a certain degree.

Further, in the conventional technology disclosed in Non-patent Document 4, the solution is stirred by unifying the two channels through which two types of solutions flow and by thereafter passing a channel of a zigzag shape therethrough. However, for sufficiently stirring the solution, it is required to pass through the zigzag portion by a long distance, and hence the size becomes spatially large. In addition, a stirred object is produced after flowing through the zigzag-shaped channel, and hence samples to be flowed are required more than a certain degree. In addition, a desired stirring cannot be achieved unless a speed of flowing through the channel is controlled according to viscosity of the solution and the zigzag shape. Therefore, the flow speed is required to be controlled with high accuracy.

Further, in the conventional technology disclosed in Non-patent Document 5, though it is the same as the conventional technology disclosed in Non-patent Document 4, in order to improve efficiency of the stirring and to shorten the portions of the zigzag-shaped channel to a certain degree, a middle portion of the zigzag-shaped channel is limited to a channel of 200 μm to 25 μm. However, it is not easy to arrange the channel of 25 μm with high accuracy.

Therefore, this invention has been made in view of the above-mentioned problems in the conventional technologies, and an object thereof is to provide a sample processing device for a microchip which has a simple and compact structure, is reduced in size and cost, and is highly-reliable.

### Means to Solve the Problems

In order to achieve the above-mentioned object, a sample processing device for a microchip of this invention includes: a sample vessel for packing a sample therein; and a reaction vessel which is continuous with the sample vessel through a channel, and to which the sample is sequentially delivered to be packed and mixed therein, and the sample is repeatedly delivered between the sample vessel and the reaction vessel through the channel so that the sample is stirred and mixed.

### EFFECT OF THE INVENTION

According to this invention, a mechanism of the sample processing device for a microchip is simplified and compactified. Further, efficient extraction of a micro component is enabled even from a minute amount of sample, and hence consumption of the expensive sample is reduced, which leads to reduction in analysis cost. Further, shortening of time required for delivery (solution-delivery) and extraction is enabled, and hence work efficiency can be considerably improved.

### BRIEF DESCRIPTION OF THE DRAWING

FIG. 1 is a perspective view illustrating a structure of a sample processing device for a microchip of this invention and a diagram of a logic circuit.

FIG. 2 is a perspective view illustrating a mechanism structure of a microchip according to this invention.

FIG. 3 is a perspective view of a partial cross-section of the microchip which is in an initial state according to this invention.

FIG. 4 is a perspective view of the partial cross-section of the microchip which is in an operation state of a first stage according to this invention.

FIG. 5 is a perspective view of the partial cross-section of the microchip which is in an operation state of a second stage according to this invention.

FIG. 6 is a perspective view of the partial cross-section of the microchip which is in an operation state of a fourth stage according to this invention.

FIG. 7 is a perspective view of the partial cross-section of the microchip which is in an operation state of a fifth stage according to this invention.

FIG. 8 is a perspective view of the partial cross-section of the microchip which is in an operation state of a sixth stage according to this invention.

FIG. 9 is a perspective view of the partial cross-section of the microchip which is in an operation state of a seventh stage according to this invention.

FIG. 10 is a perspective view of the partial cross-section of the microchip which is in an operation state of an eighth stage according to this invention.

FIG. 11 is a perspective view of the partial cross-section of the microchip which is in an operation state of a ninth stage according to this invention.

FIG. 12 is a perspective view of the partial cross-section of the microchip which is in an operation state of a tenth stage according to this invention.

FIG. 13 is a perspective view of the partial cross-section of the microchip which is in an operation state of a twelfth stage according to this invention.

FIG. 14 is a perspective view of the partial cross-section of the microchip which is in the operation state of the twelfth stage according to this invention.

FIG. 15 is a flow chart illustrating the operations of this invention.

FIG. 16 is a perspective view illustrating a mechanism structure of another microchip according to this invention.

### BEST MODE FOR EMBODYING THE INVENTION

Hereinafter, embodiments of a sample processing device for a microchip according to this invention are described in detail with reference to the drawings.

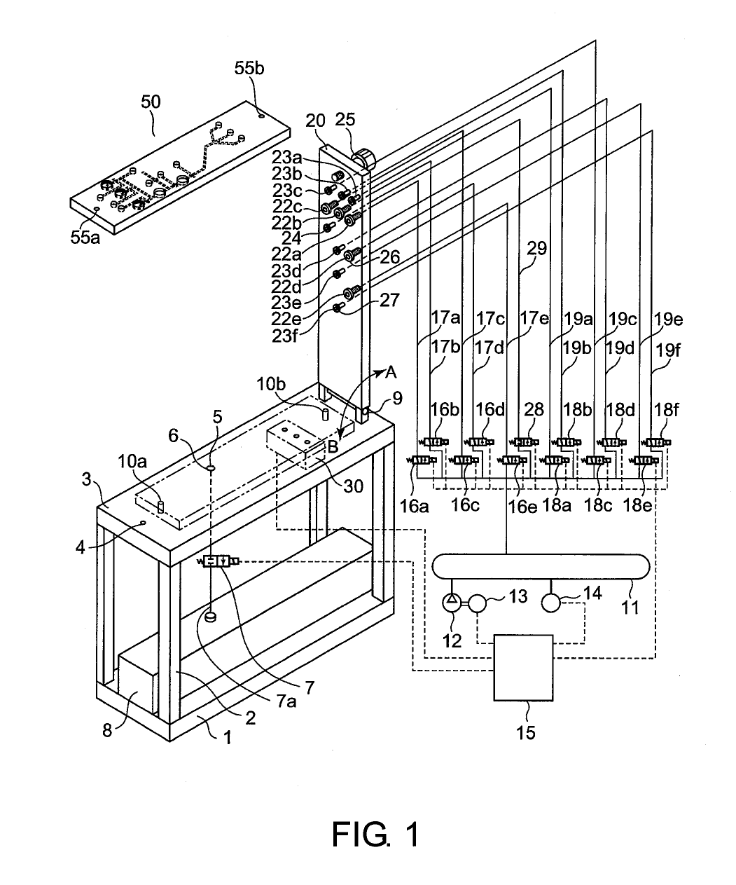

FIG. 1 is a perspective view illustrating a structure of a mechanism using the microchip of this invention to react and extract a sample in an analysis device using the microchip. Note that pneumatic circuit portions are indicated by logical symbols based on JIS.

On a machine casing 1, a table 3 is provided through poles 2. Further, in a table 3, a disposal hole 5 whose periphery is sealed by an O-ring 6 is provided. Further, the disposal hole 5 is connected to a disposal reservoir 8 provided onto the machine casing 1 through a disposal solenoid-controlled valve 7 and a tube 7a. Further, in an upper surface of the table 3, pins 10a and 10b corresponding to pin holes 55a and 55b provided in a microchip 50 to serves as a guide to a predetermined position are provided in a protruding manner. Further, on the table 3, through a hinge 9, there is provided, so as to be rotatable to the directions A and B, a cover 20 having a fastening screw 25, pressurizing holes 22a, 22b, 22c, 22d, and 22e which pass through the cover 20 and is sealed by an O-ring 26 from the peripheries thereof, shutter pressurizing holes 23a, 23b, 23c, 23d, 23e, and 23f similarly sealed by O-ring 27 from the peripheries thereof, and an air supplying hole 24 similarly sealed by the O-ring 27. Further, in one end on the table 3, a screw hole 4 is provided at a position corresponding to the fastening screw 25.

Further, the pressurizing holes 22a, 22b, 22c, 22d, and 22e which are provided while passing through the cover 20 are electrically connected to secondary sides of pressurizing solenoid-controlled valves 16a, 16b, 16c, 16d, and 16e through tubes 17a, 17b, 17c, 17d, and 17e. Further, shutter pressurizing holes 23a, 23b, 23c, 23d, 23e, and 23f are connected to secondary sides of shutter solenoid-controlled valves 18a, 18b, 18c, 18d, 18e, and 18f through tubes 19a, 19b, 19c, 19d, 19e, and 19f. Further, the air supply tube 24 is connected to the secondary side of an air supply solenoid-controlled valve 28 through a tube 29. Primary sides of the pressurizing solenoid-controlled valves 16a, 16b, 16c, 16d, and 16e, the shutter solenoid-controlled valves 18a, 18b, 18c, 18d, 18e, and 18f, and the air supply solenoid-controlled valve 28 are connected to a pressure accumulator 11. To the pressure accumulator 11, a pump 12 driven by a motor 13 and a pressure sensor 14 for detecting inner pressure are connected. Further, on the table 3, there is provided a temperature adjusting unit 30 for controlling a predetermined portion of the microchip 50 from the lower surface thereof to a predetermined temperature.

Meanwhile, to a controller 15 for executing a predetermined program, there are connected, so as to operationally controlled, the pressurizing solenoid-controlled valves 16a, 16b, 16c, 16d, and 16e, the disposal magnetic hole 7, the shutter solenoid-controlled valves 18a, 18b, 18c, 18d, 18e, and 18f, and the air supply solenoid-controlled valve 28. Further, to the controller 15, the motor 13 and the pressure sensor 14 are connected, the motor 13 driving the pump 12 so as to control the pressure in the pressure accumulator 11 to a predetermined pressure, and the pressure sensor 14 detecting the pressure in the pressure accumulator 11 to perform feedback. With the above-mentioned structure, due to instructions from the controller 15, the pressure in the pressure accumulator 11 is constantly kept in a predetermined pressure. Further, in this structure, the temperature adjusting unit 30 is similarly connected to the controller 15, to thereby perform a temperature control programmed in advance.

In this case, the air is described as an example of a medium mediating pressure. However, the same effects can be obtained as long as a material capable of mediating pressure (for example, gas, liquid, gel) is used, and hence, this invention is not limited to compressed air.

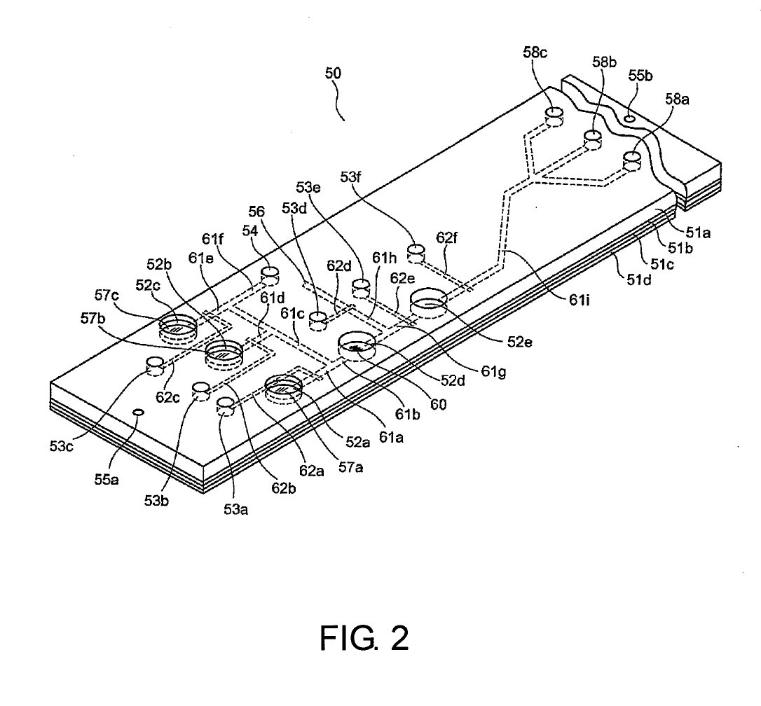

FIG. 2 is a perspective view illustrating details of the microchip 50.

The microchip 50 has a multi-layer structure, in which a main plate 51a, a second plate 51b, a third plate 51c, and a fourth plate 51d, each being made of a flexible resin, are laminated together.

On the microchip, there are provided sample reservoirs 52a, 52b, and 52c which pass through the main plate 51a and the second plate 51b to be formed into recessed shapes, and is packed with the sample in advance, and an air supply port 54. Further, there are provided a reaction reservoir 52d, an extraction reservoir 52e, and a PCR amplification reservoirs 58a, 58b, and 58c each passing through the main plate 51a to be formed into recessed shapes. Further, on the microchip 50, there are provided shutter ports 53a, 53b, 53c, 53d, 53e, and 53f passing through the main plate 51a, the second plate 51b, and the third plate 51c to be formed into recessed shapes. Further, a chip disposal hole 56 is provided so as to pass through the second plate 51b, the third plate 51c, and the fourth plate 51d to a lower direction.

Further, when the microchip 50 is installed on the table 3 illustrated in FIG. 1, and the cover 20 is rotated to a B direction, to thereby sandwich the microchip 50 between the table 3 and the cover 20 by the fastening screw 25 and the screw hole 4, the sample reservoirs 52a, 52b, and 52c, the reaction reservoir 52d, the extraction reservoir 52e, and the shutter ports 53a, 53b, 53c, 53d, 53e, and 53f are installed at positions corresponding to the pressurizing holes 22a, 22b, and 22c, the pressurizing hole 22d, the pressurizing hole 22e, and the shutter pressurizing holes 23a, 23b, 23c, 23d, 23e, and 23f, respectively.

Further, the sample reservoirs 52a, 52b, and 53c, the reaction reservoir 52d, the extraction reservoirs 52e, PCR amplification reservoirs 58a, 58b, and 58c, and the air supply port 54 are continuous with each other through channels 61a, 61b, 61c, 61d, 61e, 61f, 61g, 61h, and 61i formed between the main plate 51a and the second plate 51b. Further, shutter ports 53a, 53b, 53c, 53d, 53e, and 53f are continuous with shutter channels 62a, 62b, 62c, 62d, 62e, and 62f, respectively, which are formed between the second plate 51b and the third plate 52c. Further, leading ends thereof are provided so as to intersect the channels 61a, 61b, 61c, 61d, 61e, 61f, 61g, 61h, and 61i through the third plate 51c.

Further, the channels 61a, 61b, 61c, 61d, 61e, 61f, 61g, 61h, and 61i are formed by, when the second plate 51b and the third plate 51c are bonded to each other, not bonding portions for the channels and by keeping a separable state thereof. Similarly, the shutter channels 62a, 62b, 62c, 62d, 62e, and 62f are formed by, when the third plate 51c and the fourth plate 51d are bonded to each other, not bonding portions for the channels and by keeping the separable state thereof.

Further, the second plate 51b and the third plate 51c inside the recessed vessel of the reaction reservoir 52d and the extraction reservoirs 52e are also not bonded to each other, to thereby be continuous with the channels 61a, 61b, 61c, 61d, 61e, 61f, 61g, 61h, and 61i. Further, in an unbonded portion formed between the second plate 51b and the third plate 51c inside the reaction reservoir 52d, an adsorption member 60 for extracting a desired micro component is solid-phased.

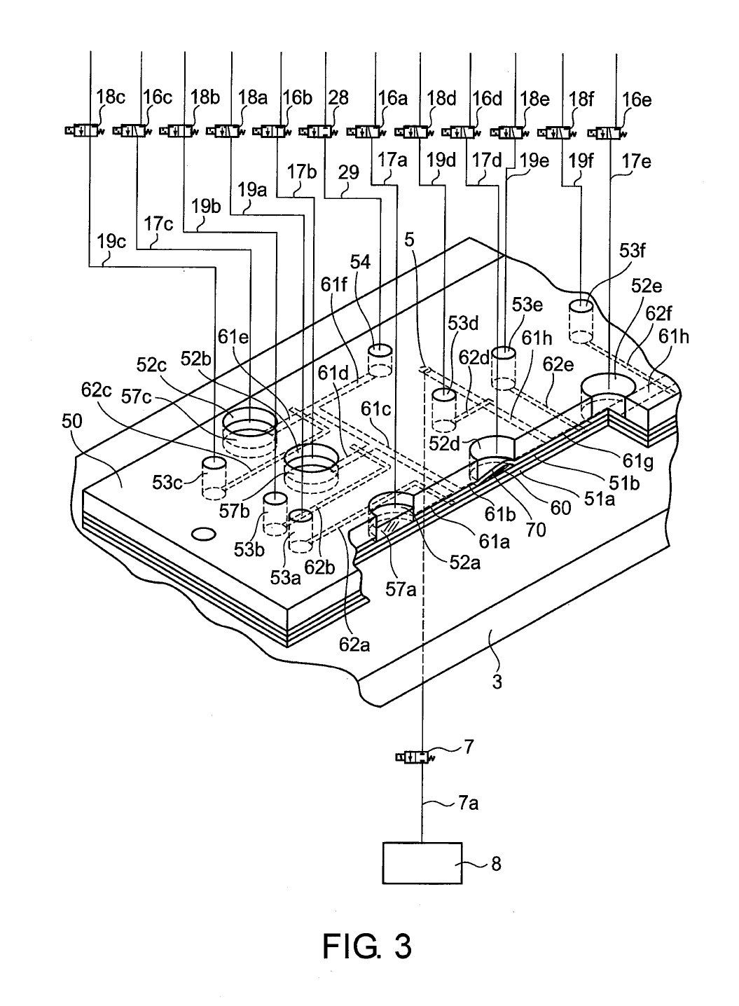

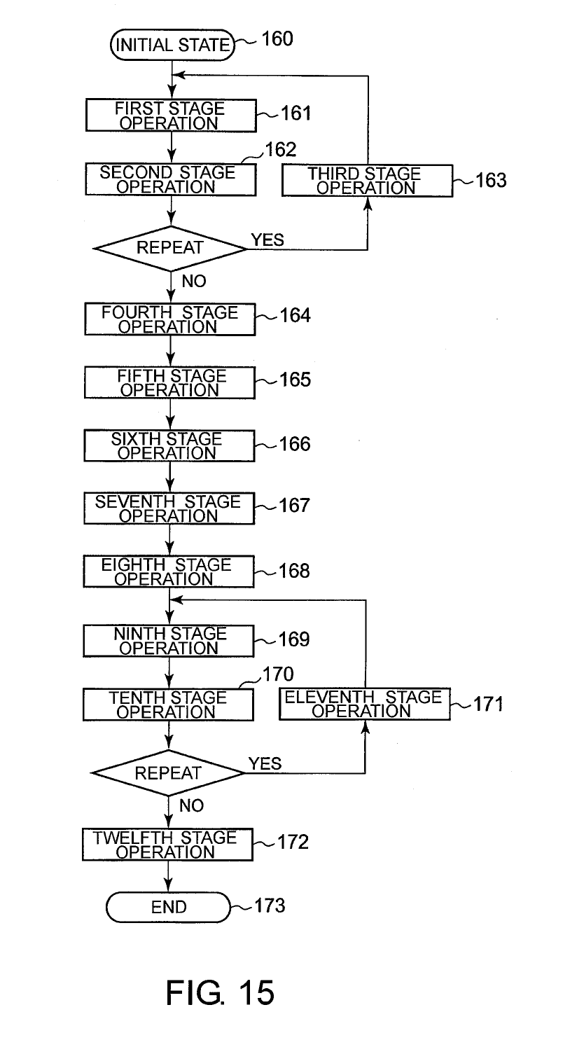

Next, operations are described with reference to FIG. 3 to FIG. 13 and a flowchart of FIG. 15.

FIG. 3 is a perspective view illustrating an initial state (step 160 in FIG. 15) of the operation, which illustrates a state in which the microchip 50 is installed on the table 3 and sandwiched by rotating the cover 20 illustrated in FIG. 1 to the B direction.

In FIG. 3, for illustrating the operations, the cover 20 and the O-rings 26 and 27 illustrated in FIG. 1 are omitted and a partial cross-section is illustrated. In the initial state, the pressurizing solenoid-controlled valves 16a, 16b, 16c, 16d, and 16e, the shutter solenoid-controlled valves 18a, 18b, 18c, 18d, 18e, and 18f, a supply electromagnet 28, and the disposal solenoid-controlled valve 7 are turned OFF. That is, the tubes 17a, 17b, 17c, 17d, and 17e, a tube 29, and the tubes 19a, 19b, 19c, 19d, 19e, and 19f are not supplied with pressurized air. As a result, the sample reservoirs 52a, 52b, and 52c, the reaction reservoir 52d, and the extraction reservoir 52e are not pressurized from above. Further, the shutter ports 53a, 53b, 53c, 53d, 53e, and 53f and the shutter channels 62a, 62b, 62c, 62d, 62e, and 62f are also not supplied with the pressurized air. Further, the air supply port 54 is also not pressed from above. Meanwhile, a circuit connected to the disposal reservoir 8 from the disposal hole 5 through the tube 7a is also shut off by the disposal solenoid-controlled valve 7.

Further, the sample reservoirs 52a, 52b, and 52c are packed with samples 57a, 57b, and 57c. Further, in the reaction reservoir 52d, there is formed a reaction chamber 70 which is a flexible unbonded portion between the second plate 51b and the third plate 51c. In the reaction chamber 70, the adsorption member 60 is solid-phased. The size of the reaction chamber 70 substantially corresponds to the diameter of the reaction reservoir 52d.

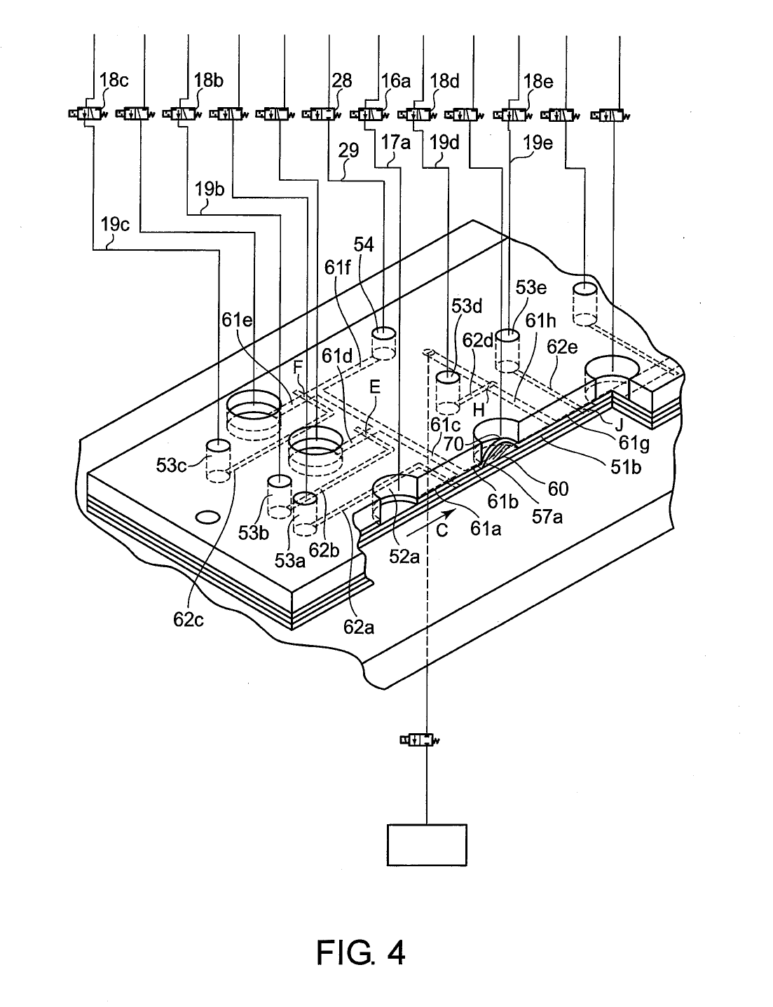

Next, a step of a first stage (FIG. 15, step 161) is described with reference to FIG. 4.

The purpose of the first stage is to deliver (solution-delivery) the sample 57a packed in the sample reservoir 52a to the reaction reservoir 52d. When the pressurizing solenoid-controlled valve 16a is turned ON from the initial state, the compressed air is guided through the tube 17a to the upper part in the sample reservoir 52a. As a result, the sample 57a extends the channel 61a to be extruded into a C direction. Further, the sample 57a also flows into the channels 61c, 61b, 61d, 61e, and 61f continuous with each other. Further, when the shutter solenoid-controlled valves 18b and 18c are turned ON, the compressed air is guided to the channels 62b and 62c through the tubes 19b and 19c and the shutter ports 53b and 53c. The channels 62b and 62c are guided below the channels 61d and 61e, and intersects therewith at portions E and F.

Therefore, the compressed air guided to the channels 62b and 62c close the channels 61d and 61e at the portions E and F, and hence, the sample 57a flowing into the channel 61c does not flow into the sample reservoirs 52b and 52c. Further, the sample 57a flowing into the channel 61f is closed because the air supply solenoid-controlled valve 28 is turned OFF and the air accumulated in the air supply port 54 is not allowed move anywhere. Further, the sample 57a flowing into the channels 61a also flows into secondary side channels 61g and 61h of the reaction reservoir 52d. However, the shutter solenoid-controlled valves 18d and 18e are turned ON, and the compressed air is introduced into the shutter channels 62d and 62e through the tubes 19d and 19e, and the shutter ports 53d and 53e, and hence, the channels 61g and 61h are closed at intersecting portions H and J with the channels 61g and 61h.

As a result, the sample 57a extruded from the sample reservoir 52a is accumulated in the reaction chamber 70 in the reaction reservoir 52d. Therefore, the upper part of the reaction chamber 70 is formed of the second plate 51b made of the flexible material, and hence the reaction chamber 70 swells like a balloon, and the sample 57a is accumulated therein. In the reaction chamber 70 in the reaction reservoir 52d, the adsorption member 60 is slid-phased in advance and adsorbs a desired micro component contained in the sample 57a. However, generally, forced stirring operation is not performed inside the reaction chamber 70, and hence adsorption efficiency is low.

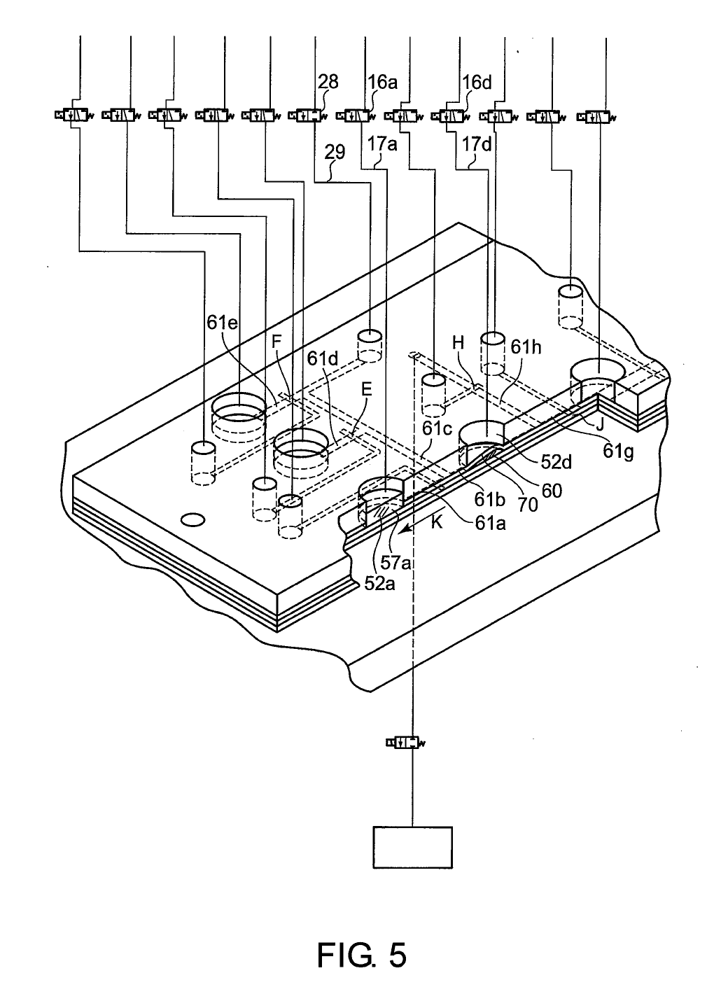

Next, a step of a second stage (step 162 in FIG. 15) are described with reference to FIG. 5.

The object of the second stage is to return the sample 57a delivered to and packed in the reaction chamber 70 in the reaction reservoir 52d at the first stage, back to the sample reservoir 52a. After the first stage is finished, when the pressurizing solenoid-controlled valve 16a is turned OFF, the sample reservoir 52a is opened to the atmosphere through the tube 17a. Further, when the pressurizing solenoid-controlled valve 16d is turned ON, the reaction reservoir 52d is pressurized through the tube 17d. As a result, the sample 57a in the reaction chamber 70 is extruded into the channels 61b, 61a, 61c, 61d, 61e, 61g, and 61h. However, as described in the operation at the first stage, the channels 61d, 61c, 61e, 61g, and 61h are closed at the intersecting portions E, F, H, and J. Further, the air supply solenoid-controlled valve 28 is turned OFF and the air in the tube 29 is closed, and hence the extruded sample 57a is guided in the channels 61a which is exclusively opened to the atmosphere to a K direction to be returned to the reservoir 52a.

Next, steps at a third stage (step 163 in FIG. 15) is described.

The object of the third stage is to reciprocate the sample 57a between the sample reservoir 52a and the reaction chamber 70 in the reaction reservoir 52d. The number of times of repetition of the first stage and the second stage is programmed in advance by the controller 15 as illustrated in the flow chart of FIG. 15. In the third stage, the first stage described with reference to FIG. 4 and the second stage as illustrated in FIG. 5 are repeated. As a result, every time the sample 57a containing the desired micro component reciprocates, the sample 57a is stirred many times by the adsorption member 60 solid-phased to the reaction chamber 70, and the desired micro component are efficiently adsorbed to the adsorption member 60. The state after the predetermined repetitions are finished in the third stage is the state illustrated in FIG. 4.

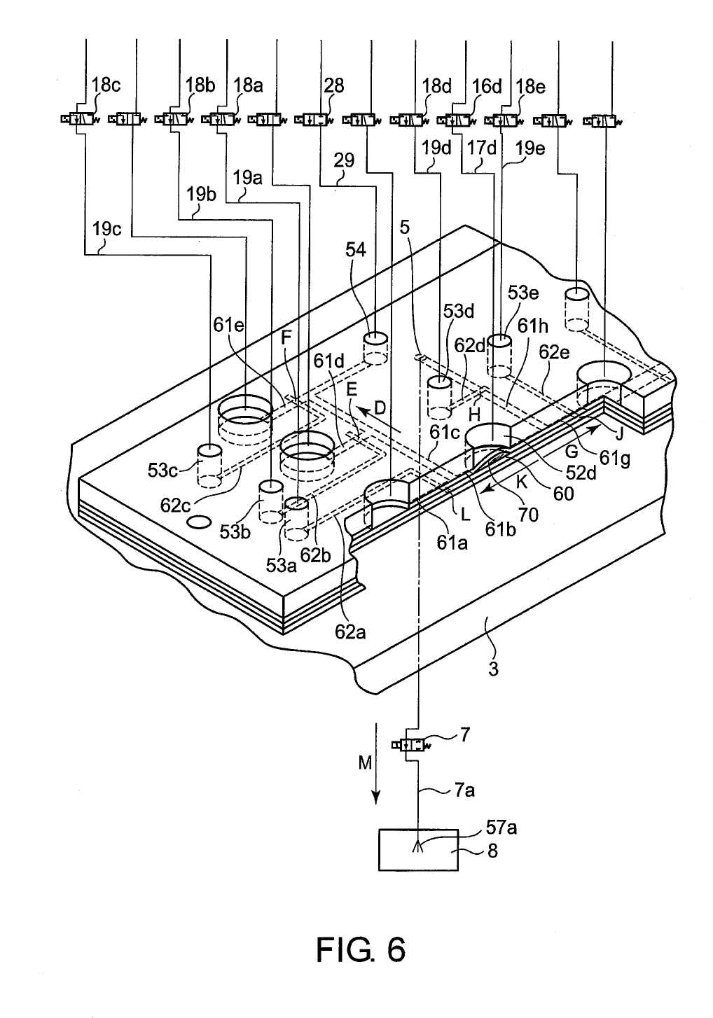

Next, a step of a fourth stage (step 164 in FIG. 15) is described with reference to FIG. 6.

The object of the fourth stage is to discharge the sample 57a in the reaction chamber 70 from the state in which the third stage illustrated in FIG. 4 is finished. Operation after the step of the third stage is finished is illustrated in FIG. 6.

The shutter solenoid-controlled valve 18a, the pressurizing solenoid-controlled valve 16d, and the disposal solenoid-controlled valve 7 are turned ON. As a result, the compressed air is guided to the reaction reservoir 52d thorough the tube 17d, and the upper part of the reaction chamber 70 is pressurized to extrude the sample 57a packed therein to the K and G directions. The extruded sample 57a flows into the channels 61b and 61c, respectively. However, the shutter solenoid-controlled valve 18a is turned ON, the compressed air is guided to the shutter channel 62a through the tube 19a and the shutter port 53a, and the shutter solenoid-controlled valves 18b and 18c are already turned ON, and hence, through the tubes 19b and 19c and the shutter ports 53b and 53c, the compressed air is supplied to the shutter channels 62b and 62c. Further, at the intersecting portions L, E, and F between the channels 61a, 61d, and 61e and the shutter channels 62a, 62b, and 62c, the sample 57a flowing into the channel 61c is blocked. Further, the air supply solenoid-controlled valve 28 is turned OFF, and hence the tube 29 and the air supply port 54 are closed in the circuit. As a result, the sample 57a guided in the channel 61c to the D direction is closed. Meanwhile, regarding the sample 57a guided in the channel 61g to the G direction, the channel 61g is blocked at the intersecting portion J with the shutter channel 62e, because the shutter solenoid-controlled valve 18e is already turned ON and the compressed air is introduced through the tube 19e and the shutter port 53e into the shutter channels 62e. Further, regarding the sample 57a guided to an I direction into the channel 61h branched from the channel 61g, because the shutter solenoid-controlled valve 18d is turned OFF, and the tube 19d, the shutter port 53d, and the shutter channel 62d are opened to the atmosphere, the channel 61h is opened at the intersecting portion H between the channel 61h and the shutter channel 62d. Further, the disposal solenoid-controlled valve 7 is turned ON, and hence the channel 61h is opened to the disposal reservoir 8 through the disposal hole 5 passing through the table 3, and the tube 7a.

With the above-mentioned structure, the sample 57a extruded from the reaction chamber 70 in the reaction reservoir 52d is guided to a M direction through the channels 61g and 61h, the disposal hole 5, the disposal solenoid-controlled valve 7, and the tube 7a, to be disposed of in the disposal reservoir 8. As a result, in the reaction chamber 70, the adsorption member 60, that adsorbs the desired micro component contained in the reagent 57a, and a part of the sample 57a containing impurities are remained.

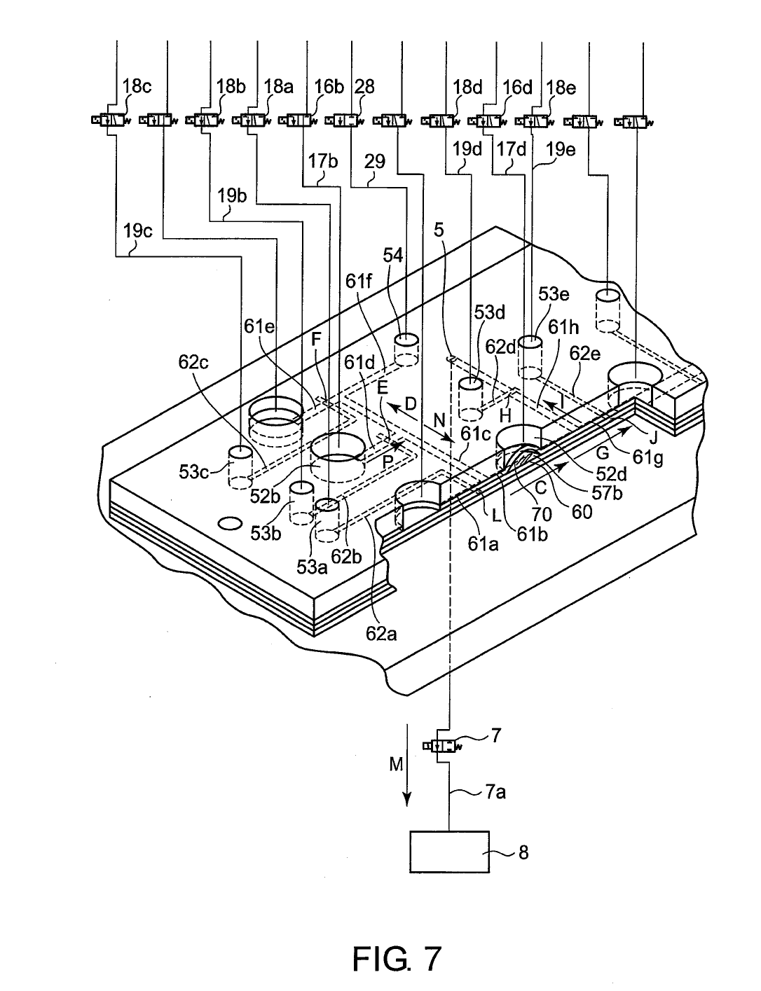

Next, a step of the fifth stage (step 165 in FIG. 15) are described with reference to FIG. 7.

The object of the fifth stage is to deliver the sample 57b illustrated in FIG. 2 into the reaction chamber 70, to thereby discharge, to the outside, impurities (components other than especially desired component) contained in the sample 57a simultaneously with the subsequent step of the sixth stage. As the sample 57b, organic solvent is generally used.

After the fourth stage is finished, the pressurizing solenoid-controlled valve 16b and the shutter solenoid-controlled valve 18d are turned ON, and the shutter solenoid-controlled valve 18b and the disposal solenoid-controlled valve 7 are turned OFF. As a result, the shutter channel 62b is opened to the atmosphere, and the portion E at which the channel 61d and the shutter channel 62b intersect with each other is opened. Further, the pressurizing solenoid-controlled valve 16b is turned ON, and hence the compressed air is guided through the tube 17b to the sample reservoir 52b, and the sample 57b packed therein is extruded to the P direction of the channel 61d. The sample 57b extruded into the channels 61d flows in the continuous channel 61c to D and N directions. However, regarding the D direction, the shutter solenoid-controlled valve 18c is turned ON, the compressed air is guided to the shutter channel 62c through the tube 19c and the shutter port 53c, and an intersecting portion F with the channel 61e is closed. Further, in the channel 61f continuous with the channel 61c, the air supply solenoid-controlled valve 28 is turned OFF and the air in the tube 29 and the air supply port 54 are sealed, and hence the sample 57b does not flow to the D direction.

Further, the sample 57b extruded to the N direction is extruded into the continuous channels 61a and 61b. However, regarding the channel 61a, the shutter solenoid-controlled valve 18a is turned ON, and the compressed air is guided to the shutter port 53a and the shutter channel 62a and is closed at the intersecting point L with the channel 61a. Therefore, the sample 57b guided to the channel 61c is guided to C direction in the channel 61b which is exclusively opened, and flows into the reaction chamber 70 in the reaction reservoir 52d. Meanwhile, though the sample 57b is also guided to G and I directions of the channels 61g and 61h continuous with the reaction chamber 70, the sample 57b does not flow into the channels 61g and 61h because the channel 61h continuous with the channel 61g is closed by the shutter solenoid-controlled valve 18d, the tube 19d, the shutter port 53d, and the shutter channel 62d at the intersecting portion H, and the shutter solenoid-controlled valve 18e is turned ON so that the compressed air is guided through the tube 19e and the shutter port 53e to the shutter channel 62e to close the intersecting portion J with the channel 61g.

As a result, similarly to the first stage, the sample 57b extruded from the sample reservoir 52b is accumulated by swelling of the reaction chamber 70 in the reaction reservoir 52d.

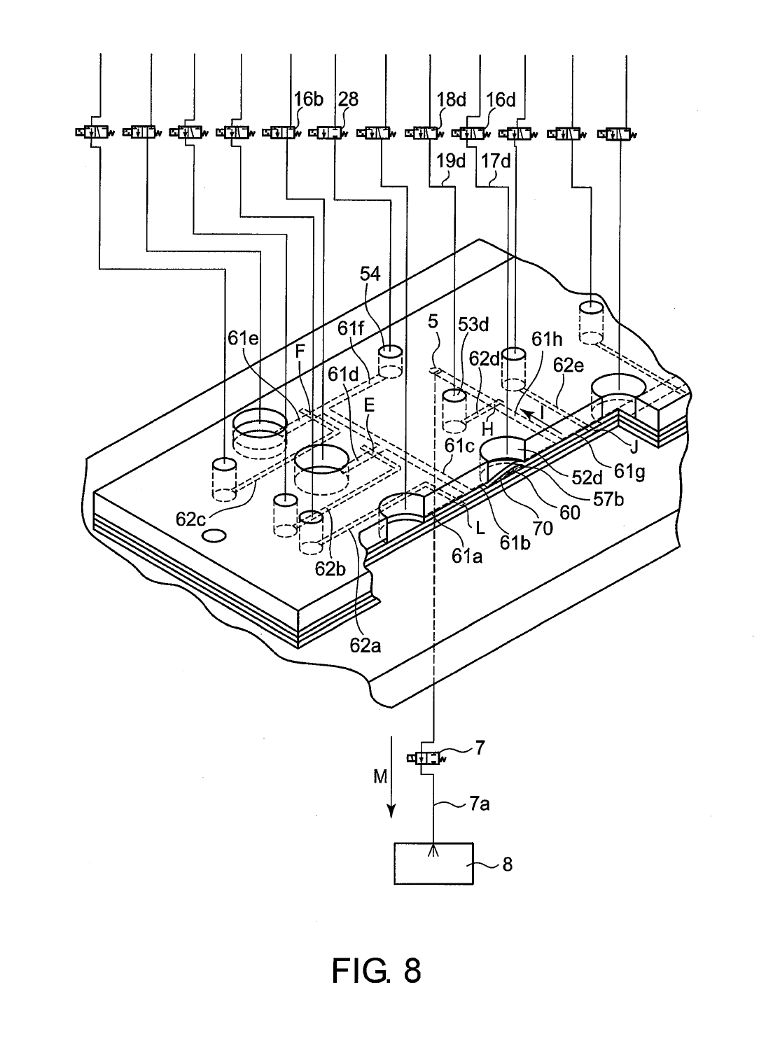

Next, a step of a sixth stage (step 166 in FIG. 15) are described with reference to FIG. 8.

The object of the sixth stage is to dispose of the sample 57b accumulated in the reaction chamber 70 in the fifth stage. After the fifth stage is finished, the pressurizing solenoid-controlled valve 16d and the disposal solenoid-controlled valve 7 are turned ON, and the pressurizing solenoid-controlled valve 16b and the shutter solenoid-controlled valve 18d are turned OFF. As a result, the compressed air is guided to the pressurizing solenoid-controlled valve 16d and the tube 17d, and the reaction chamber 70 packed with the sample 57b in the reaction reservoir 52d is compressed and the sample 57b is extruded. Further, the intersecting portions L, E, F, and J between the channels 61a, 61d, 61e, and 61g and the shutter channels 62a, 62b, 62c, and 62e are already closed, the air supply solenoid-controlled valve 28 is turned OFF, and hence a space, into which the air in the air supply port 54 and the channel 61f flows, is closed. Further, regarding the channel 61h, the shutter solenoid-controlled valve 18d is turned OFF, and the air in the tube 19d and the shutter port 53d is opened to the atmosphere. As a result, the sample 57b packed in the reaction chamber 70 is guided to the channel 61h to the I direction in which the intersecting portion H of the shutter channel 62d is exclusively opened. Further, the disposal solenoid-controlled valve 7 is turned ON, and hence the sample 57b is disposed of to the M direction through the channel 61h, the disposal hole 5, the disposal solenoid-controlled valve 7, and the tube 7a, that is, into the disposal reservoir 8.

As a result, by the reagent 57b, for which the organic solvent is generally used, impurities (for example, micro components other than desired micro component) remained in the channels 61b, 61c, and 61h and the reaction chamber 70 are flushed away. Further, the desired micro component adhered to the adsorption member 60 in the reaction chamber 70 remains.

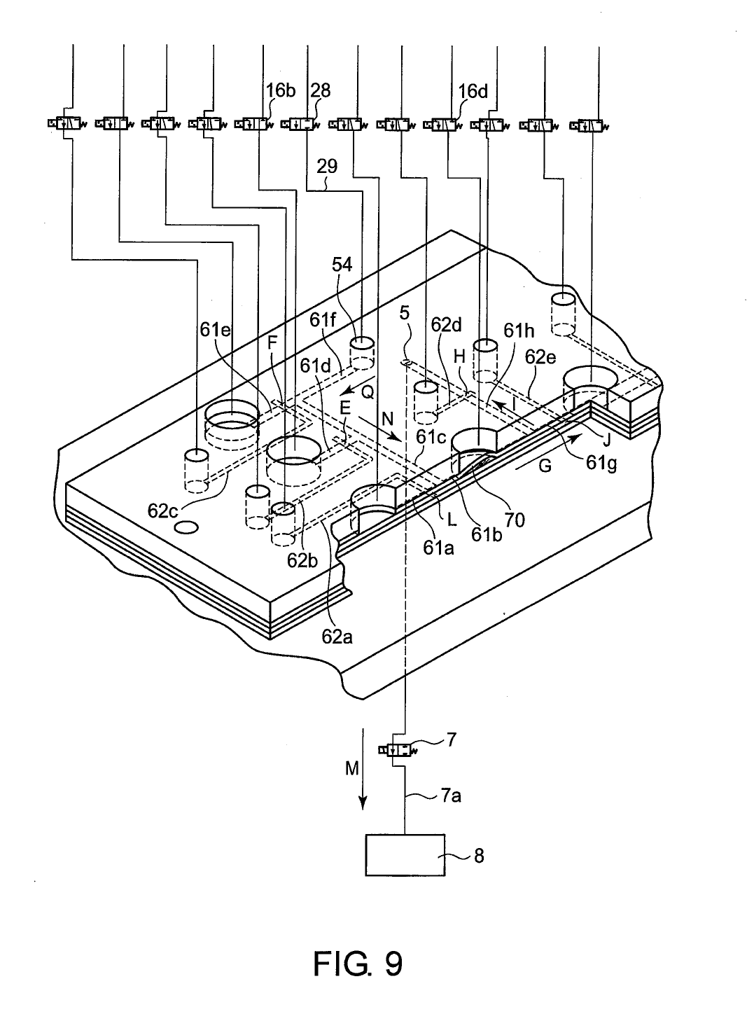

Next, a step of a seventh stage (step 167 in FIG. 15) are described with reference to FIG. 9.

Generally, as the sample 57b disposed of in the sixth stage, organic solvent is used, and it is known that a trouble is caused in the subsequent step of dissolving and extracting a desired gene (DNA) adhered to the adsorption member 60. The object of a step of the seventh stage is to volatilize and dry the channels 61b, 61c, 61f, 61g, and 61h to which the sample 57b adheres.

Operation in the seventh stage is described with reference to FIG. 9.

After the sixth stage is finished, the pressurizing solenoid-controlled valves 16b and 16d are turned OFF, and the air supply solenoid-controlled valve 28 is turned ON. Then, the compressed air is guided to a Q direction in the channel 61f through the air supply solenoid-controlled valve 28, the tube 29, and the air supply port 54. Further, the intersecting portions L, E, and F between the channels 61a, 61d, and 61e and the shutter channels 62a, 62b, and 62c and the intersecting portion J between the channel 61g and the shutter channel 62e are closed, and the intersecting portion H between the channel 61h and the shutter channel 62d is opened in the above-mentioned step of the sixth stage. Therefore, the compressed air guided to the Q direction of the channel 61f is guided to a circuit exclusively opened, that is, the channels 61f, 61c, and 61b, the reaction chamber 70, and the channels 61g and 61h to the Q, N, G, and I directions. Further, the compressed air is guided to the M direction. That is, the compressed air is guided to the disposal reservoir 8 through the disposal hole 5, and the already turned-ON disposal solenoid-controlled valve 7, and the tube 7a.

By the above-mentioned operation, the sample 57b adhered to the channels 61c and 61b, the reaction chamber 70, and the channels 61g and 61h are volatilized and dried at the sixth stage.

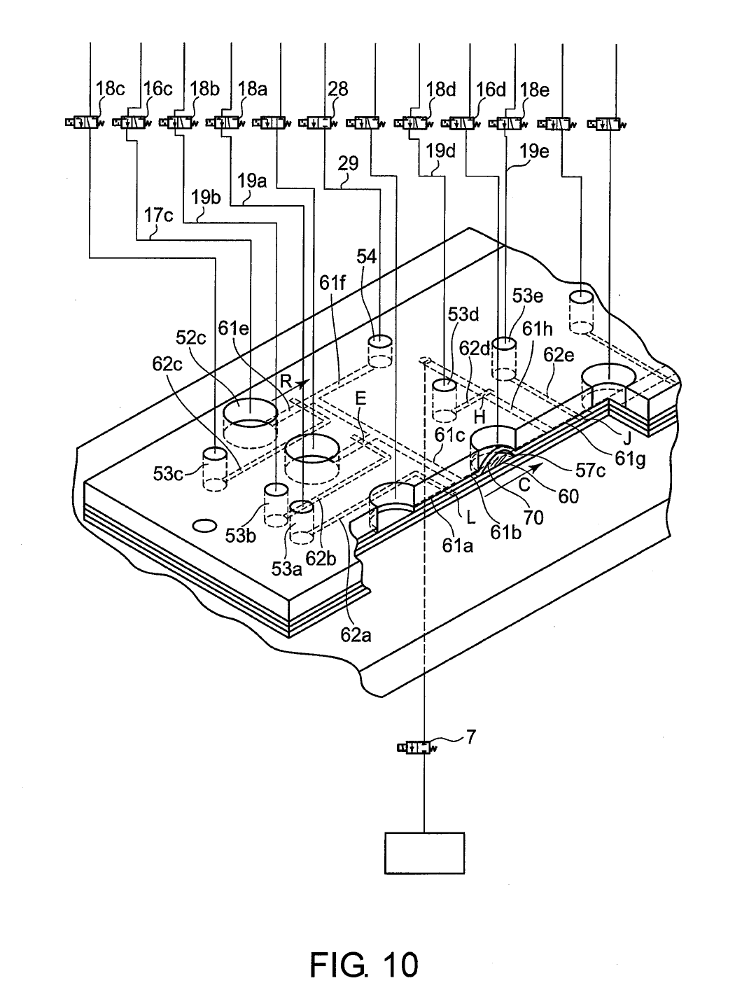

Next, a step of an eighth stage (step 168 in FIG. 15) are described with reference to FIG. 10.

The object of the eighth stage is to deliver the sample 57c packed in the sample reservoir 52c illustrated in FIG. 1 into the reaction chamber 70, to thereby dissolve and extract the desired micro component adhered to the adsorption member 60. After the step of the seventh stage is finished, the shutter solenoid-controlled valve 18c, the air supply solenoid-controlled valve 28, and the disposal solenoid-controlled valve 7 are turned OFF, and the pressurizing solenoid-controlled valve 16c and the shutter solenoid-controlled valve 18d are turned ON. When the pressurizing solenoid-controlled valve 16c is turned ON, the compressed air is guided to the sample reservoir 52c through the tube 17c, and extrudes the sample 57c into the channel 61e to an R direction, and further guides the sample 57c to the continuous channels 61c and 61f. Meanwhile, regarding the channel 61f, the air supply solenoid-controlled valve 28 is turned OFF and the air in the tube 29 and the air supply port 54 is sealed and hence the air does not flow into the channel 61f. Further, regarding the channels 62a and 62d, the shutter solenoid-controlled valves 18a and 18b are turned ON, and hence the compressed air is supplied to the tubes 19a and 19b and the shutter ports 53a and 53b, and the shutter channels 62a to 62b. Therefore, the intersecting portions L and E with the channels 61a and 61d are closed, and hence the sample 57c guided to the channel 61c flows into the channel 61b, which is exclusively opened, to the C direction.

Meanwhile, the channel 61g and the channel 61h are closed at the intersecting portions H and J with the channel 61g and the channel 61h because the shutter solenoid-controlled valves 18d and 18e are turned ON and the compressed air is supplied to the tubes 19d and 19e, the shutter ports 53d and 53e, and the shutter channels 62d and 62e. Further, the pressurizing solenoid-controlled valve 16d is turned OFF and the upper part of the reaction chamber 70 is opened to the atmosphere, and hence the sample 57c guided to the channel 61b swells the reaction chamber 70 and flows therein. The sample 57c flowing therein dissolves the desired micro component adsorbed in the reaction chamber 70 by the adsorption member 60.

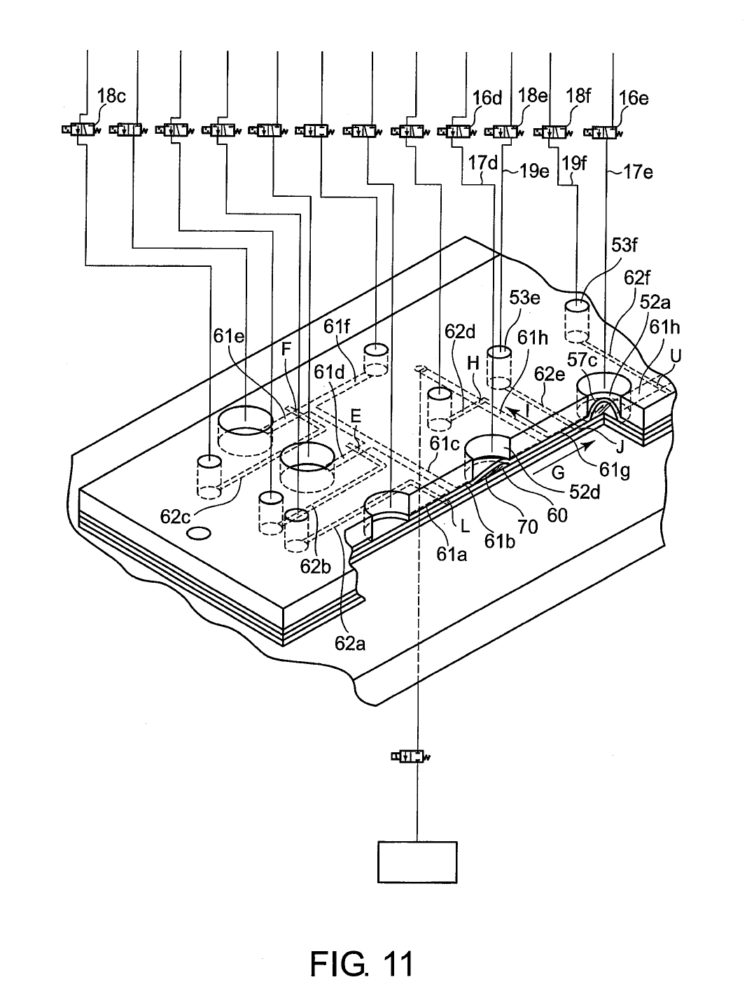

Next, a step of a ninth stage (step 169 in FIG. 15) is described with reference to FIG. 11.

The ninth stage is a step for delivering the sample 57c packed in the reaction chamber 70 in the eighth stage to the extraction reservoir 52e. After the eighth stage is finished, the pressurizing solenoid-controlled valve 16d and the shutter solenoid-controlled valves 18c and 18f are turned ON, and the shutter solenoid-controlled valve 18e is turned OFF. When the pressurizing solenoid-controlled valve 16d is turned ON, the compressed air is supplied through the tube 17d to the upper part of the reaction chamber 70 in the reaction reservoir 52d. As a result, the sample 57c in the reaction chamber 70 is extruded. However, in the eighth stage, the intersecting portions L, E, and F between the channels 61a, 61d, and 61e and the shutter channels 62a, 62b, and 62c are already closed, and the air in the channel 61f is sealed and the intersecting portion H between the channel 61h and the shutter channel 62d is also closed. Further, the shutter solenoid-controlled valve 18e is turned OFF, the shutter channel 62e is opened to the atmosphere through the tube 19e and the shutter port 53e, and the intersecting portion J between the channel 61g and the shutter channel 62e is opened. Further, when the shutter solenoid-controlled valve 18f is turned ON, the compressed air is guided to the tube 19f, the shutter port 53f, and the shutter channel 62f, and the intersecting portion U between the channel 61i and the shutter channel 62f is closed.

As a result, the sample 57c is guided in the channel 61g, which is exclusively opened, to the G direction. Further, the upper part of the extraction reservoir 52e having the same structure as the reaction chamber 70 is opened to the atmosphere through the tube 17e because the pressurizing solenoid-controlled valve 16e is turned OFF. As a result, the sample 57c whose desired micro component is dissolved in the reaction chamber 70 swells the extraction reservoir 52e like a balloon and flows and is packed therein.

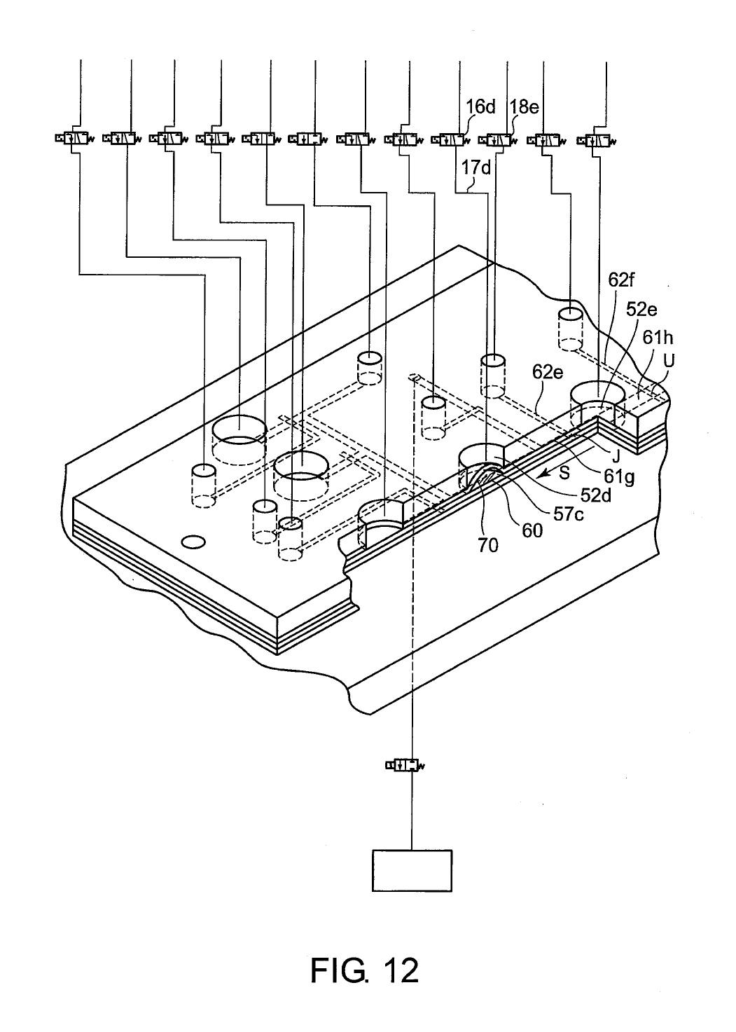

Next, a step of a tenth stage (step 170 in FIG. 15) is described with reference to FIG. 12.

It is also possible to deliver the sample 57c obtained in the extraction reservoir 52e in the above-mentioned ninth stage, in which the desired micro component is dissolved, to the PCR amplification reservoirs 58a, 58b, and 58c illustrated in FIG. 2 for the subsequent step. However, generally, if the adsorption member 60 and the sample 57c described in the eighth stage are merely brought into contact with each other, it is impossible to efficiently dissolve the desired micro component adsorbed by the adsorption member 60. Therefore, the object of the tenth stage is, similarly to the second stage, to return the sample 57c packed in the extraction reservoirs 52e to the reaction chamber 70 again, to thereby increase chances for contact between the sample 57c and the adsorption member 60 so that elution (dissolution) efficiency of the desired micro component is increased.

After the ninth stage is finished, the pressurizing solenoid-controlled valve 16d is turned OFF, and the pressurizing solenoid-controlled valve 16e is turned ON. Then, the compressed air pressurizes the extraction reservoir 52e through the tube 17e, and the upper part of the reaction reservoir 52d is opened to the atmosphere through the tube 17d, to thereby extrude the sample 57c in the extraction reservoir 52e to an S direction in the channel 61g. Further, already in the ninth stage, the intersecting portion J between the shutter channel 62e and the channel 61g is opened, and the intersecting portion U between the shutter channel 62f and the channel 61i is closed. As a result, similarly to the ninth stage, the sample 57c swells the reaction chamber 70 like a balloon and returns therein. As a result, the sample 57c returning through the channel 61g to the S direction, that is, to the reaction chamber 70, comes in contact again with the adsorption member 60, to thereby elute (dissolve) again the desired component.

As described above, by repeating the operations of the ninth stage and the tenth stage, it is possible to efficiently dissolve the desired micro component, which is adsorbed by the adsorption member 60, in the sample 57c.

Next, a step of an eleventh stage (step 171 in FIG. 15) are described.

The object of the eleventh stage is to efficiently dissolve the desired micro component adsorbed by the adsorption member 60 by repeating operation illustrated in FIG. 11 of the ninth stage and the operation illustrated in FIG. 12 of the tenth stage. The sample 57c is repeatedly reciprocated by being stirred with the adsorption member 60 in the reaction chamber 70, and hence it is possible to perform more efficient elution (dissolution) of a DNA. Further, the eleventh stage is finished in the state illustrated in FIG. 11.

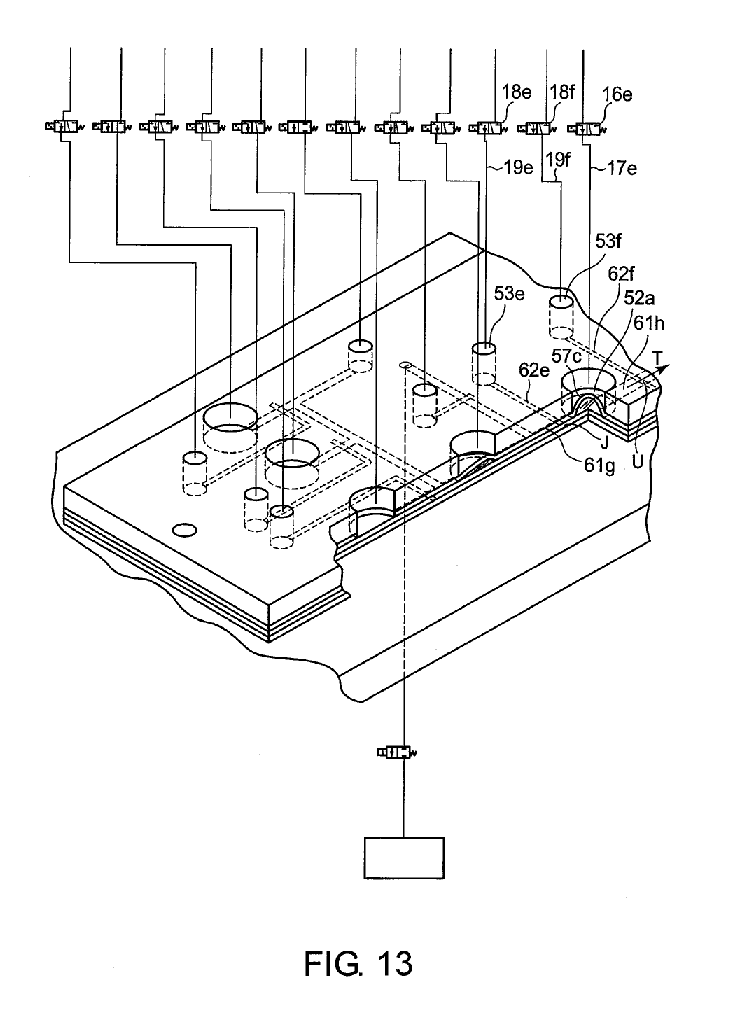

Next, a step of the twelfth stage (step 172 in FIG. 15) are described with reference to FIG. 13.

The object of the step of the twelfth stage is to deliver, to the PCR amplification reservoirs 58a, 58b, and 58c illustrated in FIG. 2 for performing the subsequent process, the sample 57c in the state after the eleventh stage in finished, that is, the sample 57c which is packed in the extraction reservoir 52e and whose desired component is dissolved.

Operation in the twelfth stage is described with reference to FIG. 13.

From the state illustrated in FIG. 11 in which the eleventh stage is finished, the pressurizing solenoid-controlled valve 16e and the shutter solenoid-controlled valve 18e are turned ON, and further the shutter solenoid-controlled valve 18f is turned OFF. As a result, the pressurizing solenoid-controlled valve 16e supplies, through the tube 17e, the compressed air to the upper part of the extraction reservoir 52e, and extrudes the sample 57c packed in the extraction reservoir 52e into the channels 61g and 61i. Meanwhile, the shutter solenoid-controlled valve 18e is turned ON, and the compressed air is supplied through the tube 19e and the shutter port 53e to the shutter channel 62e. Therefore, the intersecting portion J between the channel 61g and the shutter channel 62e is blocked, and the shutter solenoid-controlled valve 18f is turned OFF, and hence the shutter channels 62f is opened to the atmosphere through the tube 19f and the shutter port 53f, and the intersecting portion U with the channel 61i is opened.

As a result, the sample 57c in the extraction reservoir 52e is extruded to a T direction through the channel 61i which is exclusively opened. That is, the sample 57c guided to the channel 61i is delivered to the PCR amplification reservoirs 58a, 58b, and 58c illustrated in FIG. 2 for performing the subsequent step.

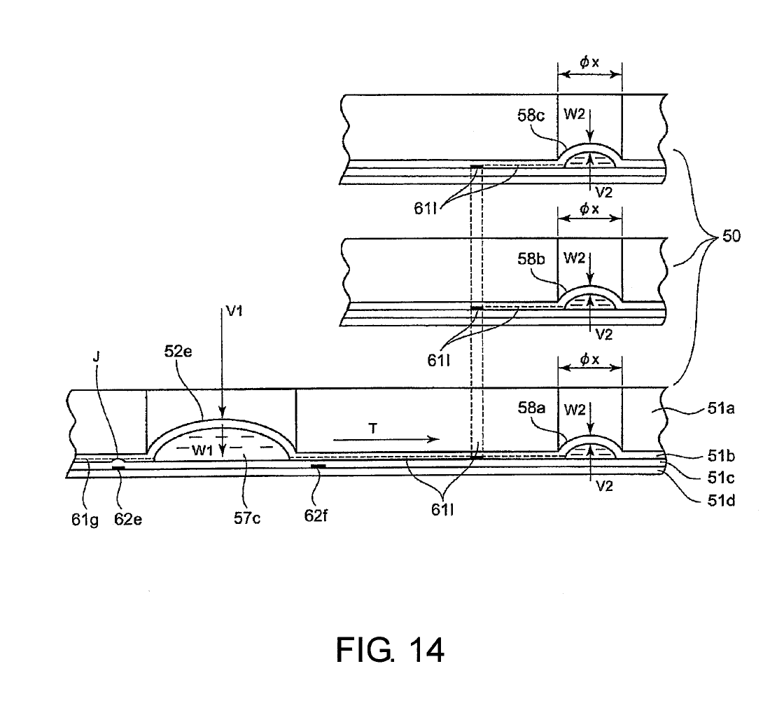

Further, details of a step of a twelfth stage (step 172 in FIG. 15) is described with reference to FIG. 14.

For the sake of convenience in description, FIG. 14 is illustrated in the form of cross-sectional view, and cross-sections of the PCR amplification reservoirs 58a, 58b, and 58c provided so as to be flush with the microchip 50 are additionally illustrated in the upper part. Further, the channels 61g and 61i and the shutter channels 62e and 62f are structurally constituted so that bonded surfaces of the second plate 51b, the third plate 51c, and the fourth plate 51d are partially formed as an unbonded structure. However, for the sake of convenience in description, the channels 61g and 61i and the shutter channels 62e and 62f are illustrated while being provided with groove-like width. As describe above, in the twelfth process, the compressed air is supplied from the upper part of the extraction reservoir 52e to a V1 direction. As a result, the sample 57c containing the desired and dissolved micro component is extruded. Further, because the compressed air is supplied to the shutter channel 62e, the channel 61g, into which the sample 57c to be flowed, on one end of the extraction reservoir 52e lifts the flexible third plate 51c constituting the shutter channel 62e in a protruding manner, and closes the shutter channel 62e at the intersecting portion J. Further, regarding the channel 61i, into which the sample 57c to be flowed, on another end of the extraction reservoir 52e, the shutter channel 62f is opened to the atmosphere. As a result, the reagent 57c in the extraction reservoir 52e is extruded to the T direction in the channel 61i which is exclusively opened. Further, the reagent 57c is guided to the PCR amplification reservoirs 58a, 58b, and 58c having the same structure as the extraction reservoirs 52e continuous with the channel 61i. Further, a force V1 extruding the sample 57c in the extraction reservoir 52e is the sum of a pressure V1 of the compressed air supplied from above and a contraction force (W1) of the flexible second plate 51b constituted by the extraction reservoir 52e (V1+W1).

Further, a force V2 of the sample 57c for swelling the PCR amplification reservoirs 58a, 58b, and 58c through channel 61i to flowing thereinto depends on a reaction force of swelling a diameter (ΦX of the flexible second plate 51b constituting the PCR amplification reservoirs 58a, 58b, and 58c. In this case, if (V1+W1)>W2 is established, logically, the reagent 57c flows into the PCR amplification reservoirs 58a, 58b, and 58c while swelling the PCR amplification reservoirs 58a, 58b, and 58c like a balloon by the force V2. Further, if the diameters (ΦX defining the PCR amplification reservoirs 58a, 58b, and 58c are equal to each other, the forces flowing therein are equal to each other, and hence swelling amounts become the same. That is, the amounts flowing into the PCR amplification reservoirs 58a, 58b, and 58c become uniform. Generally, in PCR amplification, the amplification amount is two to several μL. As a result, the minute amount of sample 57c is equally poured into the PCR amplification reservoirs 58a, 58b, and 58c.

In this manner, all steps are finished (step 173 in FIG. 15)

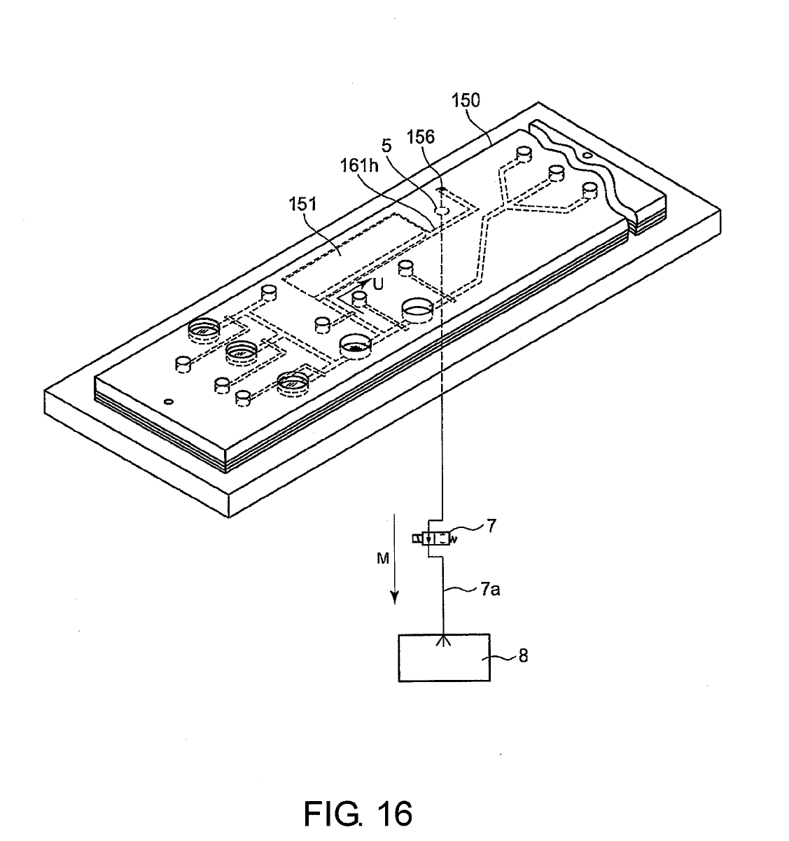

Next, a structure of another microchip is described with reference to FIG. 16.

A microchip 150 illustrated in FIG. 16 has a structure in which the above-mentioned waste solution is accumulated in the inside of the microchip 150 itself.

The waste solution disposed of toward a U direction is guided through a channel 161h to a disposal port 156. Further, similarly to the above-mentioned disposal step, the waste solution is absorbed in the disposal reservoir 8 to the M direction through the disposal solenoid-controlled valve 7 and the tube 7a. The channel 161h of the microchip 150 is opened in the channel direction toward the surface of an absorption member 151, and hence the waste solution flowing in the channel 161h changes its direction to the U direction, and hence comes into contact with the adsorption member 151, to thereby be absorbed. As a result, only gas is absorbed in the disposal reservoir 8 through the disposal solenoid-controlled valve 7 and the tube 7a. The waste solution accumulated in the microchip 150 is simultaneously disposed of when the microchip 150 is subjected to a disposal processing, and hence the disposal step is simplified.

As described above, according to the embodiments of this invention, it is possible to highly efficiently extract the desired micro component due to continuous operations from the first stage step to the twelfth stage step, that is, the adsorption operation to the adsorption member involving the stirring operation of the sample, the elimination operation of the impurities, the drying operation by the compressed air supply of the sample which becomes an obstacle for extracting the micro component, and the elution operation of the micro component involving repetitive stirring operations.

Further, according to the embodiments of this invention of this invention, the mechanism is simplified and compactified.

Further, according to the embodiments of this invention, it is possible to highly efficiently extract the micro component even from the minute amount of sample, and hence it is possible to reduce consumption of the expensive sample, to thereby reduce the analysis cost.

Further, according to the embodiments of this invention, it is possible to highly efficiently extract the micro component even from the minute amount of sample, and hence it is possible to reduce the time for solution delivery and extraction, which leads to a considerable increase of work efficiency.

Further, according to the embodiments of this invention, mixture of the micro components other than the desired components is reduced, and hence it is possible to improve reliability of the subsequent steps, that is, the amplification step and the analysis step of the micro component.

Further, according to the embodiments of this invention, it is possible to dividedly pour the sample from a single vessel to a plurality of micro vessels by a uniform amount with a simple mechanism, and hence the device can be compactified and control thereof can be simplified.

As described above, a sample processing device for a microchip of this invention includes:

a sample vessel for packing a sample therein; and

a reaction vessel which is continuous with the sample vessel through a channel, and to which the sample is sequentially delivered to be packed and mixed therein,

in which the sample is repeatedly delivered between the sample vessel and the reaction vessel through the channel so that the sample is stirred and mixed.

Preferably, the sample is repeatedly delivered so as to extract a micro component contained in the sample.

Preferably, the reaction vessel is provided with an adsorption member for extracting the micro component, and the sample is repeatedly stirred with the adsorption member while being repeatedly delivered between the sample vessel and the reaction vessel, to thereby adsorb the micro component by the adsorption member.

Preferably, a medium is supplied into the reaction vessel or the channel, to thereby dispose of the sample in the reaction vessel or the channel.

For example, a part of the sample containing impurities remains in the reaction vessel.

Preferably, the processing device further includes a second sample vessel for packing a second sample therein, and the second sample is delivered to the reaction vessel through the second channel, to thereby discharge the impurities to the outside and dispose of the second sample accumulated in the reaction vessel.

Preferably, the second sample adhered at least to the second channel and the reaction vessel is volatilized and dried.

For example, the second sample includes an organic solvent, and the second sample is volatilized and dried by compressed air.

Preferably, the sample processing device further includes a third sample vessel for packing a third sample therein, and the third sample is delivered to the reaction vessel through the third channel, to thereby dissolve the micro component, which is adsorbed by the adsorption member, in the third sample.

Preferably, the sample processing device further includes an extraction vessel, and the micro component dissolved in the third sample is delivered to the extraction vessel.

Preferably, the third sample delivered to the extraction vessel is returned to the reaction vessel so as to come into contact with the adsorption member again, to thereby dissolve the micro component in the third sample again.

A sample processing device for a microchip according to claim 11, in which a deliver operation of the micro component to the extraction vessel and a returning operation of the third sample delivered to the extraction vessel to the reaction vessel are repeated.

Preferably, the sample processing device further includes an amplification vessel for performing a desired processing, and the micro component delivered to the extraction vessel is further delivered to the amplification vessel.

Preferably, the amplification vessel includes a plurality of amplification vessels which are continuous with each other through channels branched from the extraction vessel; and the micro component is dividedly delivered to the plurality of amplification vessels by supplying a medium from an outside.

Preferably, the sample processing device further includes a disposal vessel, and the sample disposed of is contained in the disposal vessel. Alternatively, the sample disposed of is contained in the microchip.

For example, the reaction vessel, the extraction vessel, and the amplification vessels are in a state like a flexible balloon. Further, the micro component includes a gene, for example.

Hereinabove, this invention described based on the embodiments of this invention. However, it is needless to say that this invention is not limited to the above-mentioned embodiments, and various modifications can be made without departing from the gist of this invention, and such modifications are enclosed in this application.

In the above-mentioned embodiments of this invention, for the sake of convenience in description, descriptions are made while using functional appellations, such as sample reservoir, reaction reservoir, and extraction reservoir. However, appellations of the components are not limited to the above-mentioned appellations. For example, the same effects can be also obtained even when a protruding and balloon-like sample packing reservoir provided on the continuous channel is used. The balloon-like sample packing reservoir is, for example, one which is disclosed in U.S. Ser. No. 04/065,263.

Further, in the embodiments of this invention, the compressed air is used for description. However, the same effects can be obtained as long as a material capable of mediating the pressure (for example, gas, liquid, and gel) is used, and hence this invention is not limited to the compressed air. Further, if the pressurized medium is heated, it is possible to dry the object more efficiently.

This invention is based on Japanese Unexamined Patent Application Publication (JP-A) No. 2007-233574 A filed on Sep. 10, 2007, and hence contents disclosed in the above-mentioned patent application are all incorporated in this application.

1. A sample processing device for a microchip, comprising:
a sample vessel for packing a sample; and
a reaction vessel which is continuous with the sample vessel through a channel, and to which the sample is sequentially delivered to be packed and mixed,
wherein the sample is repeatedly delivered between the sample vessel and the reaction vessel through the channel so that the sample is stirred and mixed.

2. A sample processing device for the microchip according to claim 1, wherein the sample is repeatedly delivered so as to extract a micro component contained in the sample.

3. A sample processing device for the microchip according to claim 2, wherein:
the reaction vessel is provided with an adsorption member for extracting the micro component; and
the sample is repeatedly stirred with the adsorption member while being repeatedly delivered between the sample vessel and the reaction vessel, to thereby adsorb the micro component by the adsorption member.

4. A sample processing device for the microchip according to claim 1, wherein a medium is supplied into the reaction vessel or the channel, to thereby dispose of the sample in the reaction vessel or the channel.

5. A sample processing device for the microchip according to claim 4, wherein a part of the sample containing impurities remains in the reaction vessel.

6. A processing device for the microchip according to claim 5, further comprising a second sample vessel for packing a second sample,
wherein the second sample is delivered to the reaction vessel through a second channel, to thereby discharge the impurities to the outside and dispose the second sample accumulated in the reaction vessel.

7. A sample processing device for the microchip according to claim 6, wherein the second sample adhered at least to the second channel and the reaction vessel is volatilized and dried.

8. A sample processing device for the microchip according to claim 6, wherein:
the second sample comprises an organic solvent; and
the second sample is volatilized and dried by compressed air.

9. A sample processing device for the microchip according to claim 3, further comprising a third sample vessel for packing a third sample,
wherein the third sample is delivered to the reaction vessel through a third channel, to thereby dissolve the micro component, which is adsorbed by the adsorption member, in the third sample.

10. A sample processing device for the microchip according to claim 9, further comprising an extraction vessel,
wherein the micro component dissolved in the third sample is delivered to the extraction vessel.

11. A sample processing device for the microchip according to claim 10, wherein the third sample delivered to the extraction vessel is returned to the reaction vessel so as to come into contact with the adsorption member again, to thereby dissolve the micro component in the third sample again.

12. A sample processing device for the microchip according to claim 11, wherein an operation of delivering the micro component to the extraction vessel and an operation of returning the third sample, which is delivered to the extraction vessel, to the reaction vessel are repeated.

13. A sample processing device for the microchip according to claim 10, further comprising an amplification vessel for performing a desired processing,
wherein the micro component delivered to the extraction vessel is further delivered to the amplification vessel.

14. A sample processing device for the microchip according to claim 10, wherein:
the amplification vessel comprises a plurality of amplification vessels which are continuous with each other through channels branched from the extraction vessel; and
the micro component is dividedly delivered to the plurality of amplification vessels by supplying a medium from an outside.

15. A sample processing device for the microchip according to claim 4, further comprising a disposal vessel,
wherein the disposed sample is contained in the disposal vessel.

16. A sample processing device for the microchip according to claim 4,
wherein the disposed sample is contained in the microchip.

17. A sample processing device for the microchip according to claim 1, wherein the reaction vessel, the extraction vessel, and the amplification vessels are in the form of a flexible balloon.

18. A sample processing device for the microchip according to claim 1, wherein the micro component comprises a gene.

19. A sample processing device for the microchip according to claim 2, wherein a medium is supplied into the reaction vessel or the channel, to thereby dispose of the sample in the reaction vessel or the channel.

20. A sample processing device for the microchip according to claim 3, wherein a medium is supplied into the reaction vessel or the channel, to thereby dispose of the sample in the reaction vessel or the channel.
